(19) United States
(12) Patent Application Publication
(10) Pub. No.: US 2026/0165807 A1
(43) Pub. Date: Jun. 18, 2026

(54) STERILE SURGICAL DRAPE ASSEMBLY FOR 3D MOBILE C-ARM IMAGING

(71) Applicant: Tonya L McCree, Gilbert, AZ (US)

(72) Inventor: Tonya L McCree, Gilbert, AZ (US)

(21) Appl. No.: 19/383,480

(22) Filed: Nov. 7, 2025

Related U.S. Application Data
(60) Provisional application No. 63/718,541, filed on Nov. 18, 2024.

(51) Int. Cl. A61B 46/10; A61B 46/00

(52) U.S. Cl. CPC A61B 46/10; A61B 46/40

(57) ABSTRACT

A sterile surgical drape assembly specifically configured for use with three-dimensional mobile C-arm fluoroscopy systems. The drape assembly comprises upper and lower compartments temporarily joined by releasable fastening mechanism. A sterile pocket integrated into the upper compartment receives the C-arm fluoroscope housing, and an adjustable adhesive band secures the drape to the fluoroscope, ensuring the drape travels with the C-arm during rotational imaging. The design maintains sterile field integrity throughout the 3D imaging acquisition process while eliminating the need for personnel to access the area beneath the surgical table. After imaging, release tabs allow quick separation of the compartments for efficient drape removal and disposal. The invention reduces procedural time, minimizes contamination risk, decreases medical waste, and improves surgical workflow during intraoperative 3D imaging procedures.

### CROSS-REFERENCE TO RELATED APPLICATIONS

A priority claim is made to provisional patent application No. 63/718,541, filed on Nov. 8, 2024, the entire contents of which are incorporated herein by reference.

### BACKGROUND OF THE INVENTION

### 1. Field of the Invention

The present invention relates generally to surgical draping systems, and more specifically to a sterile surgical drape assembly designed for use with three-dimensional (3D) mobile C-arm fluoroscopy systems during surgical procedures requiring rotational imaging acquisition.

### 2. Description of the Related Art

Three-dimensional C-arm fluoroscopy systems have revolutionized intraoperative imaging by providing surgeons with CT-quality three-dimensional images without requiring patient transport to a radiology department. These mobile C-arm systems rotate 190-200 degrees around the patient to acquire volumetric imaging data during surgical procedures. However, this rotational motion creates significant challenges for maintaining sterile fields during surgery.

Conventional sterile draping techniques are inadequate for 3D C-arm imaging procedures. Standard surgical drapes are designed for static imaging equipment and cannot accommodate the full rotational arc required for three-dimensional image acquisition. When C-arm systems rotate beneath the surgical table, conventional drapes either restrict the imaging equipment's movement or lose contact with sterile barriers, potentially compromising the sterile field.

Current solutions to this problem involve multiple separate drapes, complex manual manipulation by surgical staff, and often require circulating staff to reach under the surgical table during the imaging sequence. These approaches are time-consuming, inefficient, increase the risk of sterile field contamination, create unnecessary procedural delays, and generate excessive medical waste. Additionally, the need for staff to crawl beneath the surgical table to manage draping materials poses ergonomic hazards and workflow disruptions.

There exists a need in the art for a sterile draping system specifically designed to accommodate the full rotational motion of 3D mobile C-arm systems while maintaining strict sterile protocols, minimizing staff manipulation, reducing procedural time, and eliminating the need for personnel to access difficult-to-reach areas beneath the surgical table.

### BRIEF SUMMARY OF THE INVENTION

The present invention provides a sterile surgical drape assembly specifically configured for use with three-dimensional mobile C-arm fluoroscopy systems. The drape assembly addresses the limitations of conventional surgical draping by providing a single-piece, disposable sterile barrier that accommodates the full rotational motion of 3D C-arm systems while maintaining sterile field integrity throughout the imaging acquisition process.

The drape assembly comprises two compartments: a sterile upper compartment measuring approximately 88 inches in width and a lower compartment measuring approximately 41 inches in width, with both compartments extending 31 inches vertically. The compartments are temporarily joined along their length by releasable fastening mechanism, allowing them to function as a unified drape during imaging and to be quickly separated for removal after the procedure.

A sterile pocket is integrated into the upper compartment and is configured to receive the fluoroscope housing of the C-arm system. The pocket includes an adjustable band with an adhesive strip along its length. The band wraps around and secures to the C-arm fluoroscope housing, ensuring the drape travels with the C-arm as it rotates beneath the surgical table. This eliminates the need for circulating staff to access the underside of the table during imaging procedures.

The drape assembly is fabricated from transparent or semi-transparent sterile plastic material that is compatible with X-ray imaging, allowing clear visualization of the surgical field while maintaining radiolucency. The overall dimensions are sufficient to encompass a human patient, the operating table, surgical instruments, medical equipment, and accessories typically present in the surgical field.

The drape includes positioning markers to assist surgical personnel in proper placement and alignment. Three release tabs positioned along the centerline of the lower compartment enable quick separation of the compartments after imaging is complete. The design incorporates accessory clips that secure gathered drape material during the imaging sequence, ensuring an unobstructed rotational path for the C-arm system.

The inventive drape assembly provides numerous advantages over prior art solutions, including reduced procedural time, decreased risk of sterile field contamination, elimination of ergonomic hazards associated with accessing beneath the surgical table, reduced medical waste through use of a single integrated drape system, and improved workflow efficiency during 3D imaging procedures.

### BRIEF DESCRIPTION OF THE DRAWINGS

The accompanying drawings, which are incorporated in and constitute a part of this specification, illustrate embodiments of the invention and, together with the description, serve to explain the principles of the invention.

FIG. 1 is a front plan view of the sterile surgical drape assembly showing the two compartments in assembled configuration, including the pocket, band, mid-marker, and release tabs.

FIG. 2 is a detailed front view of the sterile surgical drape assembly showing specific dimensions of the two compartments and component placement.

FIG. 3 is a front plan view of the upper (top) compartment showing the pocket, band, positioning markings, and attachment tabs.

FIG. 4 is a front plan view of the lower (bottom) compartment showing the three centrally-positioned release tabs.

FIGS. 5a-5b are front plan views of the adjustable band showing the paper tab release mechanism and the adhesive surface for securing to the C-arm fluoroscope housing.

FIG. 5c is a perspective view of the adjustable band in extended configuration showing the adhesive strip.

FIG. 6 is a front plan view of the three release tabs showing the hook-and-loop fastener configuration on opposing sides.

FIG. 7a is an elevation view showing the drape assembly with the band wrapped around the C-arm fluoroscope housing in the initial positioning stage prior to 3D spin acquisition.

FIG. 7b is a plan view from above the surgical table showing the patient and C-arm draped in the initial positioning stage, with the band secured and the mid-marker and release tabs visible.

FIG. 8a is an elevation view showing the patient and surgical table fully contained within the drape assembly during the second stage of 3D spin preparation, with the band removed.

FIG. 8b is a perspective view showing the drape assembly gathered and secured with accessory clips to provide clearance for C-arm rotation during the 3D imaging sequence.

FIG. 9 is a perspective view showing the drape compartments being separated via the release tabs, allowing the drape to fall laterally to the floor for removal and disposal.

FIG. 10 is a plan view of one of the accessory clips used to secure the gathered drape material during the imaging acquisition sequence.

### DETAILED DESCRIPTION OF THE INVENTION

The following detailed description of the invention refers to the accompanying drawings. The same reference numbers in different drawings may identify the same or similar elements. The following detailed description does not limit the invention. Instead, the scope of the invention is defined by the appended claims.

### Overall Structure and Configuration

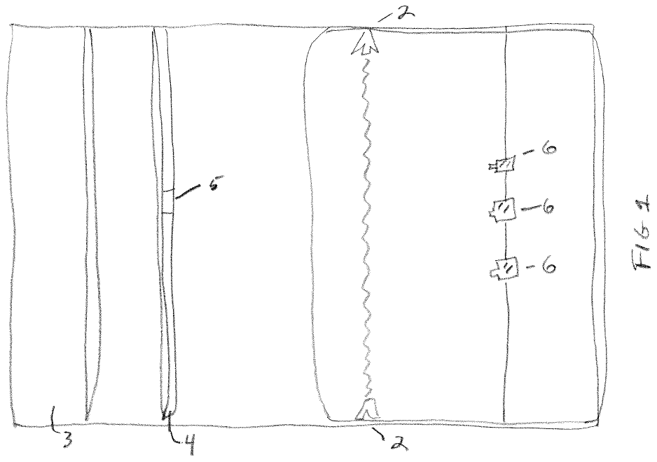

Referring to FIG. 1, the sterile surgical drape assembly of the present invention is configured as an integrated draping system designed specifically for use with three-dimensional mobile C-arm fluoroscopy systems. The drape assembly is of sufficient dimensions to encompass a human patient in anatomical position, the operating table, wires, surgical instruments, and procedural equipment including X-ray imaging devices. In the preferred embodiment, the drape measures approximately 108 inches in total width and 31 inches in height, though these dimensions may be varied to accommodate different surgical setups and equipment configurations.

The drape assembly comprises two distinct compartments: an upper sterile compartment (2) and a lower compartment (5). These compartments are temporarily joined along their longitudinal edges by releasable fastening mechanism, creating an overlapping sterile barrier that may be detached for drape removal following completion of the 3D imaging procedure. The drape is oriented perpendicular to the patient in anatomical position during use.

The drape may be fabricated from transparent or semi-transparent plastic material that maintains sterility, provides an effective barrier to contaminants, and is radiolucent to allow unobstructed X-ray imaging. The material is selected to be compatible with medical sterilization procedures and sufficiently durable to withstand the handling and manipulation required during surgical draping procedures while maintaining its barrier properties.

### Upper Compartment Structure

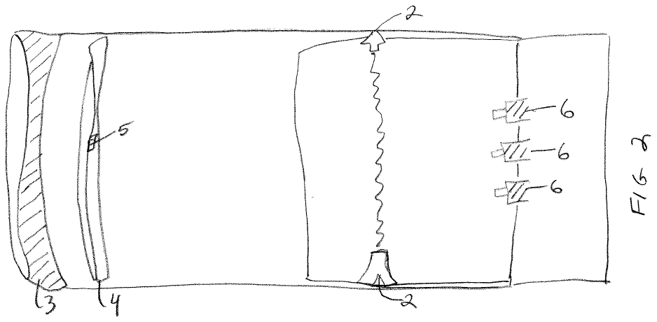

Referring to FIGS. 2 and 3, the upper compartment measures approximately 88 inches in width and 31 inches in height in the preferred embodiment. This compartment serves as the primary sterile barrier and includes several integrated components essential to the functionality of the drape assembly.

A sterile pocket (3) is attached to the surface of the upper compartment. The pocket is configured to receive and encompass the fluoroscope housing of various C-arm systems. The pocket includes a cuff measuring approximately 7 inches in width at its opening, sized to accommodate the fluoroscope housings of most 2D and 3D mobile C-arm systems currently in clinical use.

An adjustable band (4) is positioned along the top edge of the pocket, located approximately 24 inches from the outer edge of the pocket opening. The band includes an adhesive strip along its length that is protected by a removable paper release liner. When the paper liner is removed, the adhesive is exposed, allowing the band to be wrapped circumferentially around the C-arm fluoroscope housing and secured to itself. This band attachment ensures that the drape travels with the C-arm as it rotates beneath the surgical table during the imaging acquisition sequence.

The band is designed to extend and accommodate different sizes and configurations of C-arm fluoroscope housings. The adjustable nature of the band allows a single drape design to be compatible with multiple C-arm systems from different manufacturers, increasing the versatility and cost-effectiveness of the invention.

A mid-marker (2) is positioned on the upper compartment to assist with proper drape placement. When this marker is positioned near the patient's spine during draping, it indicates that adequate overlap of the compartments has been achieved to maintain sterility throughout the procedure.

### Lower Compartment Structure

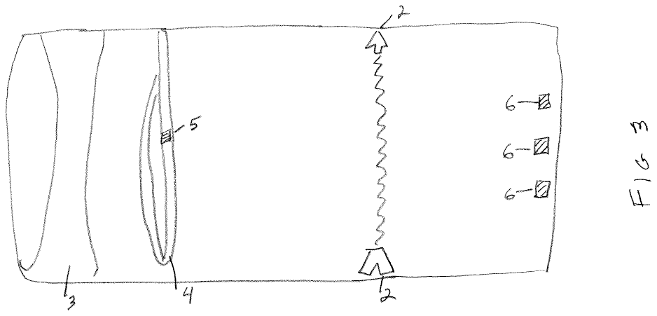

Referring to FIGS. 2 and 4, the lower compartment measures approximately 41 inches in width and 31 inches in height. This compartment is temporarily attached to the upper compartment along their overlapping longitudinal edges, creating the complete drape assembly during use.

Three release tabs (6) are positioned along the centerline of the lower compartment, spaced approximately 20-21 inches apart vertically. Each release tab comprises a two-sided fastener system, with hook fasteners on one side and loop fasteners on the opposing side. This dual-sided configuration allows the tabs to secure the overlap between the upper and lower compartments during use, while enabling quick separation when the tab is pulled to release one compartment from the other.

### Release Mechanism and Band Details

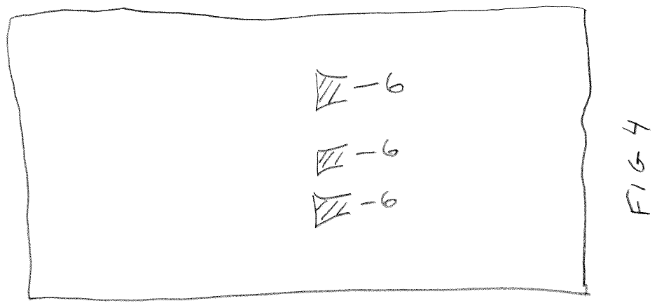

Referring to FIGS. 5a, 5b, and 5c, the adjustable band (4) includes paper sleeves or release tabs that maintain the band in a compact configuration during sterile packaging and initial handling. To deploy the band, the surgical personnel breaks the paper sleeve to access the free end of the band. The protective sticker covering the adhesive strip is then removed to expose the adhesive surface.

The band is wrapped circumferentially around the C-arm fluoroscope housing with sufficient tension to ensure secure attachment. The exposed adhesive bonds to the opposing surface of the band, creating a closed loop around the fluoroscope housing. This secure attachment allows the drape to travel under the surgical table as the C-arm moves into position for the 3D imaging sequence, eliminating the need for circulating staff to crawl beneath the table to manage the drape.

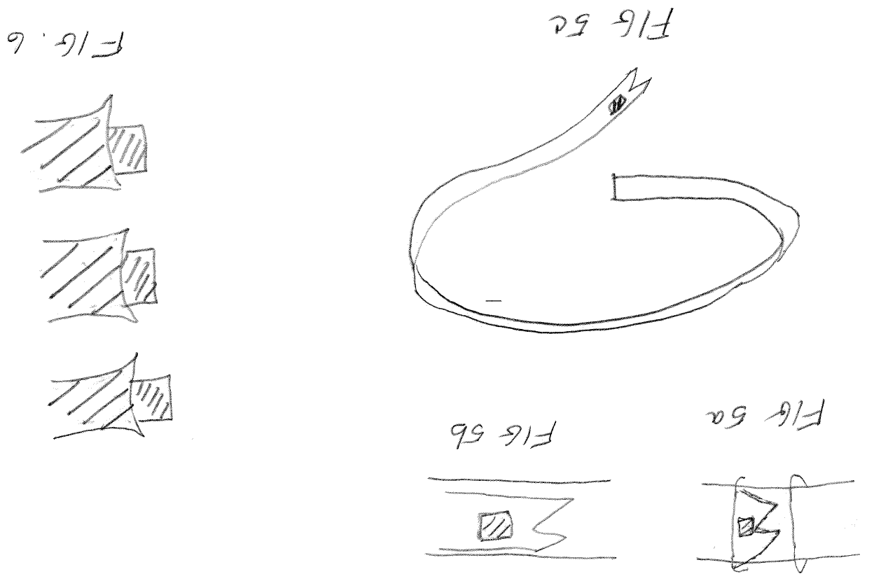

Referring to FIG. 6, the release tabs (6) are illustrated in detail. Each tab features hook fasteners on one side and loop fasteners on the opposite side. During drape assembly, the hook side of each tab on the lower compartment engages with corresponding loop material on the upper compartment (or vice versa), temporarily joining the compartments. To remove the drape after imaging, one side of each tab is pulled, causing the hook-and-loop connection to separate and allowing the two compartments to split apart.

### Method of Use

The sterile surgical drape assembly is used in conjunction with 3D mobile C-arm fluoroscopy systems according to the following method:

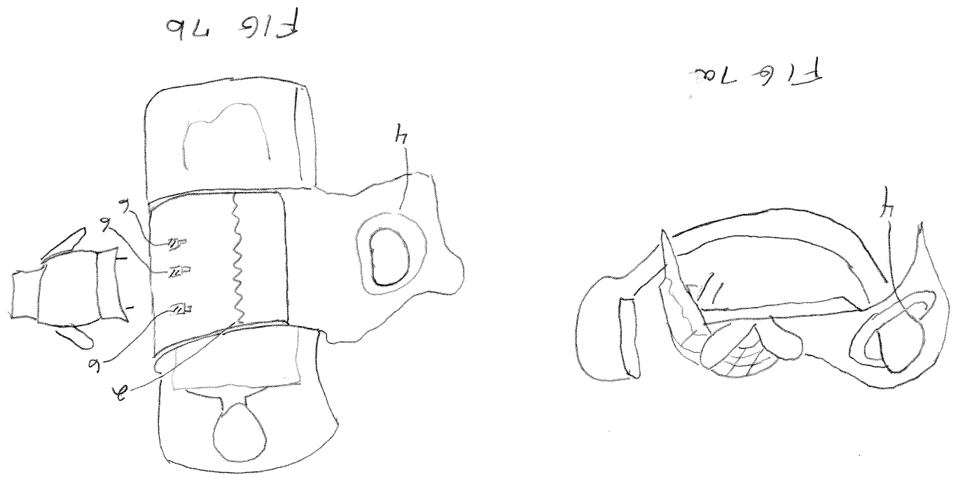

Initial Draping Phase (FIGS. 7a and 7b): The sterile surgical drape assembly is removed from its sterile packaging by sterile surgical personnel. The C-arm fluoroscope is positioned adjacent to the surgical table in preparation for imaging. Sterile personnel extend the pocket (3) of the upper compartment over the fluoroscope housing of the C-arm. The paper sleeve on the band (4) is broken to release the free end of the band, and the protective sticker is removed to expose the adhesive strip. The band is wrapped tightly around the fluoroscope housing and secured to itself via the adhesive, creating a closed loop that attaches the drape to the C-arm.

The mid-marker (2) is positioned near the patient's spine to verify proper drape placement and adequate overlap between compartments. The release tabs (6) maintain the connection between the upper and lower compartments, preserving the sterile field. At this stage, the C-arm can be rotated toward its pre-scan position, with the drape traveling automatically beneath the surgical table due to its attachment via the band.

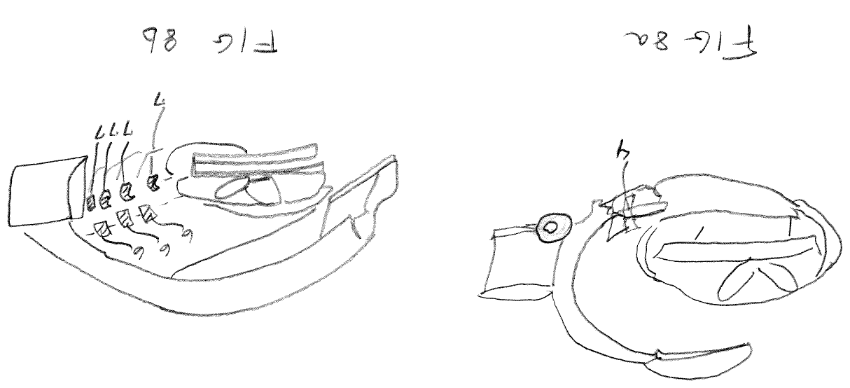

Imaging Preparation Phase (FIGS. 8a and 8b): Once the C-arm has rotated to bring the drape beneath the table and just below the sterile field, circulating personnel can access the band from the non-sterile side and release it from the fluoroscope housing. The edges of the upper and lower compartments are brought together on the non-sterile side of the surgical table. The drape material is gathered and rolled to remove excess material and create visual clearance for the C-arm's rotational path.

Four accessory clips (7), as shown in FIG. 10, are used to secure the gathered drape material. These clips are standard disposable surgical clips familiar to operating room personnel. The clips maintain the drape in a compact, secured configuration that encompasses the patient, table, IV lines, and any equipment hanging from the table, while providing unobstructed clearance for the C-arm to complete its 190-200 degree rotational scan.

Imaging Acquisition Phase: With the drape secured by the clips and positioned to provide clear rotational clearance, the C-arm system executes its 3D imaging sequence without interruption. The drape maintains the sterile field throughout the imaging process while allowing the C-arm to rotate freely around the patient.

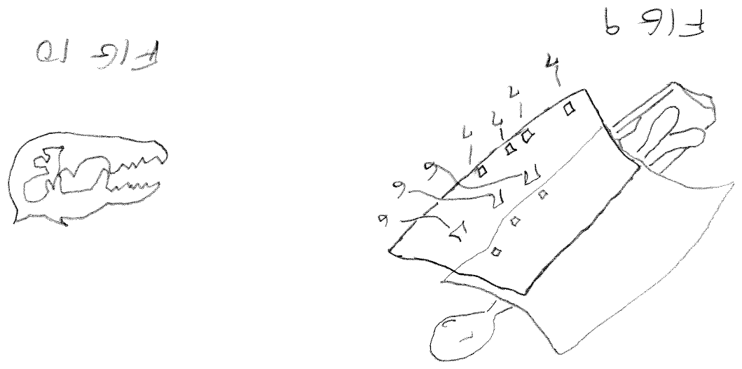

Drape Removal Phase (FIG. 9): After 3D imaging is complete, circulating personnel access the three release tabs (6) located on the non-sterile side of the patient. Each release tab is pulled to separate the hook-and-loop connection, causing the upper and lower compartments to split apart. The accessory clips (7) do not need to be removed prior to tab release. Once the tabs are released, the two compartments of the drape fall laterally away from the center of the patient to the floor. Circulating personnel then gather the drape material, pull it to the non-surgical side of the room, and dispose of it according to standard medical waste protocols.

### Advantages of the Invention

The sterile surgical drape assembly of the present invention provides numerous advantages over previous draping systems:
(1) Single Integrated System: The invention replaces multiple separate drapes with a single integrated draping system, reducing complexity, setup time, and medical waste.
(2) Elimination of Under-Table Access: The band attachment mechanism eliminates the need for circulating staff to crawl beneath the surgical table to position or retrieve draping materials, improving workflow efficiency and reducing ergonomic hazards.
(3) Maintained Sterility: The design ensures continuous maintenance of the sterile field throughout the imaging procedure, reducing the risk of surgical site contamination.
(4) Rapid Removal: The release tab mechanism allows quick, efficient removal of the drape after imaging is complete, reducing procedural time and allowing surgical personnel to resume the operation more quickly.
(5) Universal Compatibility: The adjustable band accommodates various C-arm systems from different manufacturers, making the drape suitable for use across different operating room configurations and equipment.
(6) Clear Imaging Path: The transparent material and gathered configuration ensure the X-ray beam path remains unobstructed while maintaining the sterile barrier.
(7) Simplified Training: The intuitive design requires minimal training for surgical and circulating personnel, facilitating adoption and reducing the learning curve associated with 3D imaging procedures.

### Alternative Embodiments

While the invention has been described with reference to specific embodiments, various modifications and alternative configurations are possible without departing from the scope of the invention. The dimensions provided represent a preferred embodiment but may be adjusted to accommodate different patient sizes, surgical table configurations, or C-arm system specifications. The pocket size and band length may be varied to fit different C-arm fluoroscope housing dimensions. The number and spacing of release tabs may be modified based on drape size and intended application. Alternative fastening mechanisms, such as adhesive strips, mechanical snaps, or magnetic fasteners, may be substituted for the hook-and-loop release tabs described. The drape material may comprise various medical-grade plastics, films, or fabrics that provide appropriate sterile barrier properties and radiolucency.

One skilled in the art will appreciate that many variations are possible within the scope of the claims. Thus, while the disclosure or certain preferred embodiments of the present invention are particularly shown and described above, it will be understood by those skilled in the art that these and other changes in form and details may be made therein without departing from the spirit and scope of the claims.

Unless otherwise indicated, all numbers expressing a characteristic, item, quantity, parameter, property, term, and so forth used in the present specification and claims are to be understood as being modified in all instances by the term “about.” As used herein, the term “about” mechanism that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosed embodiments (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”), provided herein is intended merely to better illuminate the present disclosure and does not pose a limitation on the scope of the embodiments otherwise claimed. No language in the present specification should be construed as indicating any non-claimed element essential to the practice of the disclosed embodiments of the present invention.

1. A sterile surgical drape assembly for use with a three-dimensional mobile C-arm fluoroscopy system, comprising:
an upper compartment fabricated from sterile, radiolucent material, the upper compartment having a width dimension of approximately 88 inches and a height dimension of approximately 31 inches;
a sterile pocket attached to a surface of the upper compartment, the pocket configured to receive a fluoroscope housing of the C-arm fluoroscopy system;
an adjustable band positioned along a top edge of the pocket, the band having an adhesive strip configured to secure the band circumferentially around the fluoroscope housing, thereby ensuring the drape assembly travels with the C-arm as the C-arm rotates beneath a surgical table during three-dimensional image acquisition;
a lower compartment fabricated from sterile material, the lower compartment having a width dimension of approximately 41 inches and a height dimension of approximately 31 inches;
releasable fastening mechanism positioned along overlapping edges of the upper compartment and the lower compartment, the releasable fastening mechanism configured to temporarily join the compartments during the image acquisition and to enable separation of the compartments for removal after the image acquisition is complete; and
a positioning marker on the upper compartment to assist with proper alignment of the drape assembly during placement.

2. The sterile surgical drape assembly of claim 1, wherein the releasable fastening mechanism comprises a plurality of release tabs positioned along a centerline of the lower compartment, each the release tab comprising hook fasteners on a first side and loop fasteners on a second side opposing the first side.

3. The sterile surgical drape assembly of claim 2, wherein the plurality of release tabs comprises three release tabs spaced approximately 20 to 21 inches apart along the centerline of the lower compartment.

4. The sterile surgical drape assembly of claim 1, wherein the pocket includes a cuff measuring approximately 7 inches in width, the cuff configured to accommodate fluoroscope housings of various C-arm systems.

5. The sterile surgical drape assembly of claim 1, wherein the adjustable band is positioned approximately 24 inches from an outer edge of the pocket.

6. The sterile surgical drape assembly of claim 1, wherein the adjustable band includes a paper release liner protecting the adhesive strip prior to use, the paper release liner being removable to expose the adhesive strip for securing the band to the fluoroscope housing.

7. The sterile surgical drape assembly of claim 1, wherein the upper compartment and the lower compartment are fabricated from transparent or semi-transparent plastic material that is radiolucent and maintains sterility.

8. The sterile surgical drape assembly of claim 1, wherein the drape assembly has overall dimensions sufficient to encompass a human patient, an operating table, surgical instruments, and medical equipment during a surgical procedure.

9. The sterile surgical drape assembly of claim 1, further comprising a plurality of accessory clips configured to secure gathered portions of the drape assembly during rotation of the C-arm, thereby providing clearance for unobstructed imaging.

10. A method of maintaining a sterile field during three-dimensional C-arm fluoroscopy imaging, comprising the steps of: a) providing a sterile surgical drape assembly comprising an upper sterile compartment having an integrated pocket with an adjustable adhesive band, and a lower compartment releasably attached to the upper compartment; b) positioning the pocket over a fluoroscope housing of a C-arm fluoroscopy system; c) wrapping the adhesive band circumferentially around the fluoroscope housing and securing the band to itself, thereby attaching the drape assembly to the C-arm; d) rotating the C-arm to position the drape assembly beneath a surgical table; e) releasing the band from the fluoroscope housing; f) gathering and securing the drape assembly with accessory clips to provide clearance for rotational motion of the C-arm; g) executing a three-dimensional imaging acquisition sequence; and h) releasing the lower compartment from the upper compartment by activating release fasteners, allowing the drape assembly to fall away from a patient for removal and disposal.

11. The method of claim 10, wherein the step of releasing the lower compartment comprises pulling a plurality of release tabs positioned along a centerline of the lower compartment, each the release tab comprising hook-and-loop fasteners that separate when pulled.

12. The method of claim 10, wherein the step of positioning the pocket includes aligning a positioning marker on the upper compartment near a spine of the patient to verify adequate overlap between the upper compartment and the lower compartment.

13. The method of claim 10, wherein the step of wrapping the adhesive band includes removing a protective paper liner from the band to expose an adhesive surface prior to securing the band around the fluoroscope housing.

14. The method of claim 10, wherein the step of rotating the C-arm causes the drape assembly to travel beneath the surgical table automatically due to attachment of the band to the fluoroscope housing, thereby eliminating need for personnel to access an area beneath the surgical table.

15. The method of claim 10, wherein the accessory clips comprise four clips positioned to secure gathered material of the drape assembly while encompassing the patient, the surgical table, intravenous lines, and equipment associated with the surgical table.

16. A sterile pocket drape system for rotational medical imaging equipment, comprising:
a primary drape panel of sterile radiolucent material having dimensions sufficient to cover a surgical patient and associated medical equipment;
a secondary drape panel of sterile material configured to overlap with the primary drape panel along a longitudinal edge;
a sterile equipment pocket affixed to the primary drape panel, the pocket configured to receive imaging equipment and having a cuff opening dimensioned to accommodate various equipment housing sizes;
a flexible band with adhesive properties attached to the pocket, the band configured to wrap around and secure to the imaging equipment, causing the drape system to move in coordination with rotational motion of the imaging equipment;
separable fastening elements positioned between the primary drape panel and the secondary drape panel, the fastening elements maintaining panel overlap during an imaging procedure and enabling rapid panel separation for drape removal; and
visual alignment indicators on the primary drape panel to facilitate proper drape positioning.

17. The sterile pocket drape system of claim 16, wherein the separable fastening elements comprise multiple release tabs each having hook fasteners on one side and loop fasteners on an opposing side, the tabs spaced along a centerline of the secondary drape panel.

18. The sterile pocket drape system of claim 16, wherein the flexible band includes a removable protective covering over the adhesive properties, and wherein the band is configured to extend to accommodate equipment housings of varying circumferences.

19. The sterile pocket drape system of claim 16, further comprising a plurality of mechanical fasteners configured to secure gathered portions of the drape system, thereby creating clearance for unobstructed rotation of the imaging equipment.

20. The sterile pocket drape system of claim 16, wherein the primary drape panel and the secondary drape panel are fabricated from transparent or semi-transparent material that provides sterile barrier properties while allowing visual assessment of underlying surgical instruments and equipment.
